(19) United States
(12) Patent Application Publication
(10) Pub. No.: US 2024/0299597 A1
(43) Pub. Date: Sep. 12, 2024

(54) 3-Iodopropyl-1,2,3-Triazol-1-yl, 3-Bromopropyl-1,2,3-Triazol-1-yl, and Derivatives for Uses in Labeling

(71) Applicant: Emory University, Atlanta, GA (US)

(72) Inventors: Mark M. Goodman, Atlanta, GA (US); W. Robert Taylor, Stone Mountain, GA (US); Wone Woo Seo, Alpharetta, GA (US); Malgorzata Lipowska, Decatur, GA (US)

(21) Appl. No.: 18/572,536

(22) PCT Filed: Jul. 1, 2022
(86) PCT No.: PCT/US2022/035922

§ 371 Date: Dec. 20, 2023

Related U.S. Application Data
(60) Provisional application No. 63/217,651, filed on Jul. 1, 2021.

(51) Int. Cl. A61K 51/06

(52) U.S. Cl. CPC A61K 51/065

(57) ABSTRACT

This disclosure relates to using 3-iodopropyl-1,2,3-triazol-1-yl or 3-bromopropyl-1,2,3-triazol-1-yl as linking groups for generating labeled conjugates. In certain embodiments, disclosure relates to using 3-iodopropyl-1,2,3-triazol-1-yl or 3-bromo-propyl-1,2,3-triazol-1-yl as linking groups for generating labeled polysaccharide conjugates or derivatives. In certain embodiments, this disclosure relates to methods of generating radionuclides.

### CROSS-REFERENCE TO RELATED APPLICATIONS

This application claims the benefit of U.S. Provisional Application No. 63/217,651 filed Jul. 1, 2021. The entirety of this application is hereby incorporated by reference for all purposes.

### BACKGROUND

Infections are currently diagnosed by using blood cultures or tissue biopsy; however, these methods can only detect late-stage infections that are challenging to treat. A major limitation preventing the effective treatment of bacterial infection is an inability to image infections in vivo with accuracy and sensitivity. Consequently, bacterial infections can be diagnosed only after they have become systematic or have caused significant anatomical tissue damage, a stage at which they are challenging to treat owing to the high bacterial burden. Although contrast agents have been developed to image bacteria, their clinical impact has been minimal because they are unable to detect small numbers of bacteria in vivo and cannot distinguish infections from other pathologies such as cancer and inflammation. There is a need for the development of contrast agents that can image small numbers of bacteria accurately in vivo.

Bacteria can utilize glycogen, starch, and amylose as carbon sources. Prior to transport through the cell membrane, these polysaccharides are hydrolyzed by the extracellular amylase into smaller maltodextrins, maltose and isomaltose. The maltose ABC importer (type I) of Escherichia coli enables the bacteria to feed on maltose and maltodextrins (Bordignon et al., Mol Microbiol., 2010, 77(6):1354-1366).
Murthy et al. report oligosaccharides conjugates for targeting bacteria. See WO/2012/097223 and WO/2016/044846.
Ning et al. report fluorine-18 labeled maltohexaose images bacterial infections by PET. Angew Chem Int Ed Engl. 2014, 53(51):14096-14101.
Zlitni et al. report maltotriose-based probes for fluorescence and photoacoustic imaging of bacterial infections. Nat Commun. 2020, 11, 1250.

References cited herein are not an admission of prior art.

### SUMMARY

This disclosure relates to using 3-iodopropyl-1,2,3-triazol-1-yl, 3-bromopropyl-1,2,3-triazol-1-yl, and derivatives as linking groups for generating labeled conjugates. In certain embodiments, disclosure relates to using 3-iodopropyl-1,2,3-triazol-1-yl or 3-bromopropyl-1,2,3-triazol-1-yl as linking groups for generating labeled polysaccharide conjugates or derivatives. In certain embodiments, this disclosure relates to methods of generating radionuclides.

In certain embodiments, the polysaccharide or derivatives may be directly linked to the with 3-iodopropyl-1,2,3-triazol-1-yl or 3-bromopropyl-1,2,3-triazol-1-yl groups or may be separated by a linking group.

In certain embodiments, this disclosure relates to compounds having the following formula:

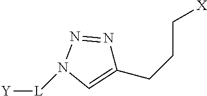

or salts thereof wherein, X is I or Br; Y is a polysaccharide or polysaccharide derivative; and L is a linking group or L is absent and Y forms a direct bond from a sugar unit in the polysaccharide to the nitrogen.

In certain embodiments, the polysaccharide is maltotriose, isomaltotriose, maltoheptaose, or maltohexaose.

In certain embodiments, the polysaccharide derivative is an acetylated polysaccharide or carboxymethylated polysaccharide.

In certain embodiments, the linking group is an alkyl group or glycol group.

In certain embodiments, the compound is 2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, the compound is 2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, the compound is 2-(acetoxymethyl)-6-((4,5-diacetoxy-2-(acetoxymethyl)-6-((4,5-diacetoxy-2-(acetoxymethyl)-6-(3-(4-(3-bromopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, this disclosure relate to uses of compounds disclosed herein for imaging, measuring, or detecting a bacterial infection. In certain embodiments, compounds disclosed herein are used to produce a labeling agent for imaging, measuring, or detecting a bacterial infection.

### BRIEF DESCRIPTION OF THE SEVERAL VIEWS OF THE DRAWING(S)

FIG. 1A illustrates the preparation of F-18 maltodextrin using a tosyl group.

FIG. 1B illustrates a proposed mechanism of the formation of 3-tosyl-propyl-1,2,3-triazol-1-yl byproduct.

FIG. 1C illustrates the production of a F-18 maltotriose using a 3-iodo-propyl-1,2,3-triazol-1-yl precursor or a 3-bromo-propyl-1,2,3-triazol-1-yl precursor. It was discovered that the iodo and bromo derivatives are more statable to storage when compared to the tosylate and 4-bromo-tosylate derivatives.

FIG. 1D illustrates the production of an iodo maltohexaose acetylated precursor compound.

FIG. 2 illustrates the structures of the iodo maltotriose acetylated precursor with the chemical name 2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate and the bromo maltotriose acetylated precursor with the chemical name 2-(acetoxymethyl)-6-((4,5-diacetoxy-2-(acetoxymethyl)-6-((4,5-diacetoxy-2-(acetoxymethyl)-6-(3-(4-(3-bromopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate.

FIG. 3 illustrates the chemical structure of the iodo maltohexaose acetylated precursor with the chemical name 2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3,4,5-triyl triacetate.

### DETAILED DESCRIPTION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

An “embodiment” of this disclosure refers to an example and infers that the example is not necessarily limited to the example. Embodiments of the present disclosure will employ, unless otherwise indicated, techniques of medicine, organic chemistry, biochemistry, molecular biology, pharmacology, and the like, which are within the skill of the art. Such techniques are explained fully in the literature. Prior to describing the various embodiments, the following definitions are provided and should be used unless otherwise indicated.

It must be noted that, as used in the specification and the appended claims, the singular forms “a,” “an,” and “the” include plural referents unless the context clearly dictates otherwise. In this specification and in the claims that follow, reference will be made to a number of terms that shall be defined to have the following meanings unless a contrary intention is apparent.

“Positron emission tomography” (PET) refers to an imaging technique that produces an image, e.g., three-dimensional image, by detecting pairs of gamma rays emitted indirectly by a positron-emitting radionuclide tracer. Images of tracer concentration within the area are then constructed by computer analysis. A radioactive tracer is administered to a subject e.g., into blood circulation. Typically, there is a waiting period while tracer becomes concentrated in areas of interest; then the subject is placed in the imaging scanner. As the radionuclide undergoes positron emission decay, it emits a positron, an antiparticle of the electron with opposite charge, until it decelerates to a point where it can interact with an electron, producing a pair of (gamma) photons moving in approximately opposite directions. These are detected in a scanning device. The technique typically utilizes simultaneous or coincident detection of the pair of photons moving in approximately opposite direction (the scanner typically has a built-in slight direction-error tolerance). Photons that do not arrive in pairs (i.e. within a timing-window) are typically ignored. One typically localizes the source of the photons along a straight line of coincidence (also called the line of response, or LOR). This data is used to generate an image.

The term “radionuclide” or “radioactive isotope” refers to molecules of enriched isotopes that exhibit radioactive decay (e.g., emitting positrons). Such isotopes are also referred to in the art as radioisotopes. A radionuclide tracer does not include radioactive primordial nuclides but does include a naturally occurring isotopes that exhibit radioactive decay with an isotope distribution that is enriched, e.g., is several fold greater than natural abundance. In certain embodiments, is contemplated that the radionuclides are limited to those with a half live of less than 1 hour and those with a half-life of more than 1 hour but less than 24 hours. Radioactive isotopes are named herein using various commonly used combinations of the name or symbol of the element and its mass number (e.g., ¹⁸F, F-18, or fluorine-18).

As used herein, the term “saccharide” refers to sugars or sugar derivatives, polyhydroxylated aldehydes and ketones, e.g., with an empirical formula that approximates Cₘ(H₂O)ₙ, i.e., wherein m and n are the same or about the same. Contemplated saccharides include, e.g., maltose, isomaltose, and lactose with an empirical formula of C₁₂H₂₂O₁₁. The term is intended to encompass sugar monomers, oligomers, and polymers. The terms oligosaccharide and polysaccharide are used interchangeably, and these saccharides typically contain between two and ten monosaccharide units, or greater than ten monosaccharide units. In certain embodiments, a polysaccharide refers to a polymer of 3 or more sugar units. In certain embodiments of the disclosure, the saccharide is a dextrin, maltodextrin, or cyclodextrin. Dextrins are mixtures of polymers of D-glucose units linked by α-(1→4) or α-(1→6) glycosidic bonds. Maltodextrin consists of D-glucose units connected in chains of variable length. The glucose units are primarily linked with α(1→4) glycosidic bonds. Maltodextrin is typically composed of a mixture of chains that vary from three to nineteen glucose units long. Maltose is a disaccharide formed from two units of glucose joined with an α(1→4) bond. Isomaltose has two glucose molecules linked through an α(1→6) bond. In certain embodiments, the disclosure contemplates cyclic and noncyclic polysaccharides. Typical cyclodextrins contain a number of glucose monomers ranging from six to eight units in a ring, such as alpha cyclodextrin; a six membered sugar ring molecule; beta cyclodextrin, a seven membered sugar ring molecule; and gamma cyclodextrin, an eight sugar ring molecule. In certain embodiments, saccharides and polysaccharides are contemplated to include bridging thiol linkages, i.e., one or more of the sugar units are glucose bridged by thiol through a 1→4 and or 1→6 bond.

As used herein, “salts” refer to derivatives of the disclosed compounds where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like. In preferred embodiment the salts are conventional nontoxic pharmaceutically acceptable salts including the quaternary ammonium salts of the parent compound formed, and non-toxic inorganic or organic acids. Preferred salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

“Subject” refers any animal, preferably a human patient, livestock, rodent, monkey, or domestic pet.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing an oxygen atom with a sulfur atom or replacing an amino group with a hydroxy group. Derivatives may be prepared by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze, hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxy, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, heteroarylalkyl.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

“Alkanoyl” refers to an alkyl as defined above attached through a carbonyl bridge (i.e., —(C═O)alkyl). Acetyl refers to an alkanoyl wherein the alkyl is methyl (i.e., —(C═O)CH₃.

A “linking group” refers to any variety of molecular arrangements that can be used to bridge to molecular moieties together. An example formula may be —Rₙ— wherein R is selected individually and independently at each occurrence as: —CRₙRₙ—, —CHRₙ—, —CH—, —C—, —CH₂—, —C(OH)Rₙ, —C(OH)(OH)—, —C(OH)H, —C(Hal)Rₙ—, —C(Hal)(Hal)-, —C(Hal)H—, —C(N₃)Rₙ—, —C(CN)Rₙ—, —C(CN)(CN)—, —C(CN)H—, —C(N₃)(N₃)—, —C(N₃)H—, —O—, —S—, —N—, —NH—, —NRₙ—, —(C═O)—, —(C═NH)—, —(C═S)—, —(C═CH₂)—, which may contain single, double, or triple bonds individually and independently between the R groups. If an R is branched with an Rₙ it may be terminated with a group such as —CH₃, —H, —CH═CH₂, —CCH, —OH, —SH, —NH₂, —N₃, —CN, or -Hal, or two branched Rs may form a cyclic structure. It is contemplated that in certain instances, the total Rs or “n” may be less than 100 or 50 or 25 or 10. Examples of linking groups include bridging alkyl groups and alkoxyalkyl groups.

A “glycol” refers to an alkyl that contains oxygen atoms at the ends of the alkyl group and is optionally a polymer (i.e., —O-[alkyl-O]ₙ—, wherein n is typically 1 to 10, 100, 1000, or more). A terminal end of the glycol may contain an alkyl or hydroxy group (e.g., —O-[alkyl-O]ₙ-alkyl or —O-[alkyl-O]ₙ—H). Polyethylene glycol is an example.

A “deacetylating agent” refers to an agent or combination of agents that transform an acetyl ester, e.g., on a polysaccharide as a derivative of a hydroxy group, into a hydroxy group. This is typically accomplished by exposing an acetyl group to aqueous basic conditions that spontaneously convert acetate esters into acetic acid; however, it is contemplated that this may be accomplished by alternative methods, such as, by exposure to enzymes such as esterases.

Certain of the compounds described herein may contain one or more asymmetric centers and may give rise to enantiomers, diastereomers, and other stereoisomeric forms that can be defined, in terms of absolute stereochemistry at each asymmetric atom, as (R)- or (S)-. The present chemical entities, compositions and methods are meant to include all such possible isomers, including racemic mixtures, tautomer forms, hydrated forms, optically substantially pure forms and intermediate mixtures. In certain embodiments, the compounds may be present in a composition with enantiomeric excess or diastereomeric excess of greater than 60%. In certain embodiments, the compounds may be present in enantiomeric excess or diastereomeric excess of greater than 70%. In certain embodiments, the compounds may be present in enantiomeric excess or diastereomeric excess of greater than 80%. In certain embodiments, the compounds may be present in enantiomeric excess or diastereomeric excess of greater than 90%. In certain embodiments, the compounds may be present in enantiomeric excess or diastereomeric excess of greater than 95%.

### 3-Iodopropyl and 3-Bromopropyl Stabilized Precursor Compounds for Radiolabeling

This disclosure relates to using 3-iodopropyl-1,2,3-triazol-1-yl or 3-bromopropyl-1,2,3-triazol-1-yl as linking groups for generating labeled polysaccharide conjugates or derivatives. In certain embodiments, this polysaccharide or derivatives may be directly linked to the with 3-iodopropyl-1,2,3-triazol-1-yl or 3-bromopropyl-1,2,3-triazol-1-yl groups or may be separated by a linking group.

In certain embodiments, this disclosure relates to a compound having the following formula:

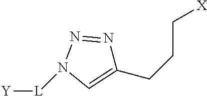

or salts thereof wherein, X is I or Br; Y is a polysaccharide or polysaccharide derivative; and L is a linking group or L is absent and Y forms a direct bond from a sugar unit in the polysaccharide to the nitrogen.

In certain embodiments, the polysaccharide is maltotriose, isomaltotriose, maltoheptaose, or maltohexaose.

In certain embodiments, the polysaccharide derivative is an acetylated polysaccharide or carboxymethylated polysaccharide.

In certain embodiments, the linking group is an alkyl group or glycol group.

In certain embodiments, this disclosure relates to a compound having the following formula:

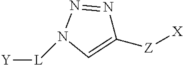

or salts thereof wherein, X is I or Br; Y is a polysaccharide or polysaccharide derivative; and L is a linking group or L is absent and Y forms a direct bond from a sugar unit in the polysaccharide to the nitrogen, and Z is a linking group, —CH₂—, —CH₂CH₂—, —CH₂CH₂CH₂—, —CH₂CH₂CH₂CH₂—, or —CH₂(CH₂)ₙCH₂—, wherein n is 2-20.

In certain embodiments, the compound is 2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, the compound is 2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, the compound is 2-(acetoxymethyl)-6-((4,5-diacetoxy-2-(acetoxymethyl)-6-((4,5-diacetoxy-2-(acetoxymethyl)-6-(3-(4-(3-bromopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

In certain embodiments, compounds are prepared as racemic mixtures or as isomers with enantiomeric or diastereomeric excess. In certain embodiments, the compounds have greater than 55%, 60%, 70%, 80%, 90%, or 95% enantiomeric excess or diastereomeric excess.

In certain embodiments, this disclosure relates to methods of generating a compound isotopically enriched with fluorine 18 comprising contacting a compound as disclosed herein with hydrofluoric acid isotopically enriched with fluorine 18 providing a compound isotopically enriched with fluorine 18. In certain embodiments, methods further comprise contacting the compound isotopically enriched with fluorine 18 with a deacetylating agent such that alkanoyl groups on compound are converted to hydroxy groups providing a polysaccharide isotopically enriched with fluorine 18. In certain embodiments, the deacetylating agent is sodium hydroxide or other a metal hydroxide in an aqueous solution or mix aqueous and ethanol solution.

In certain embodiments, this disclosure relates to methods comprising a) administering a composition comprising a polysaccharide or polysaccharide derivative isotopically enriched with fluorine 18 made by the process disclosed herein to a subject; and b) scanning the subject for emissions. In certain embodiments, the method further comprises the step of detecting the emissions and creating an image indicating or highlighting the location of the compound containing polysaccharide or polysaccharide derivative isotopically enriched with fluorine 18 in the subject.

In certain embodiments, this disclosure relate to uses of compounds disclosed herein for imaging, measuring, or detecting a bacterial infection. In certain embodiments, compounds disclosed herein are used to produce a labeling agent for imaging, measuring, or detecting a bacterial infection.

In certain embodiments, this disclosure relates to methods comprising a) administering a composition comprising a polysaccharide or polysaccharide derivative isotopically enriched with fluorine 18 made by the process disclosed herein to a subject; and b) scanning the subject for emissions. In certain embodiments, the method further comprises the step of detecting or measuring the emissions and creating an image indicating or highlighting the location of the compound containing polysaccharide or polysaccharide derivative isotopically enriched with fluorine 18 in the subject. In certain embodiments, detecting, measuring, or identifying the location of the compound in an area provides for detection of the presence of a bacterial infection at that location.

In certain embodiments, this disclosure relates to kits comprising a compound disclosed herein and optionally a substance having an isotopically enriched element for preparing a radionuclide. In certain embodiments, this disclosure relates to kits comprising a compound disclosed herein and a complex of potassium carbonate and/or potassium bound to [2.2.2]-cryptand N(CH₂CH₂OCH₂CH₂OCH₂CH₂)₃N.

In certain embodiments, this disclosure relates to methods of preparing compounds disclosed herein comprising mixing starting materials and optionally reagents under conditions such that the products are formed.

### Tracer Radionuclides

Radionuclides are isotopically labeled forms of compounds disclosed herein including isotopes of hydrogen, carbon, nitrogen, oxygen, phosphorous, technetium, fluorine, chlorine, and iodine, such as ²H, ³H, ¹¹C, ¹³C, ⁴C, ⁵N, ¹⁵O, ¹⁷O, ³¹P, ³²P, ^{99m}Tc, ³⁵S, ¹⁸F, ³⁶Cl, ¹²⁵I and ¹³¹I. It will be understood that compounds of the disclosure can be labeled with an isotope of any atom or combination of atoms in the structure. While [¹⁸F] has been emphasized herein as being particularly useful for PET, SPECT and tracer analysis, other uses are contemplated including those flowing from physiological or pharmacological properties of stable isotope homologs and will be apparent to those skilled in the art.

Such isotopically labeled compounds are useful in metabolic studies, reaction kinetic studies, detection, or imaging techniques [such as positron emission tomography (PET) or single-photon emission computed tomography (SPECT)] including drug or substrate tissue distribution assays, or in radioactive treatment of patients. In particular, an ¹⁸F or ^{99m}Tc labeled compound may be particularly preferred for PET or SPECT studies, respectively. Isotopically labeled compounds of this disclosure and prodrugs thereof can generally be prepared by carrying out the procedures disclosed in the schemes or in the examples and preparations described herein by substituting a readily available isotopically labeled reagent for a non-isotopically labeled reagent.

In certain embodiments, compounds disclosed herein are substituted with ¹⁸F, Flurone-18. Radiofluorination reactions are typically nucleophilic substitutions. Aromatic nucleophilic substitutions with fluoride usually require activated aromatic rings, bearing both a good leaving group (e.g. a halogen, a nitro- or a trimethylammonium group) and a strong electron-withdrawing substituent (e.g. a nitro-, cyano- or acyl group) preferably placed para to the leaving group, whereas aliphatic nucleophilic substitutions typically utilize leaving group (usually a halogen or a sulfonic acid derivative such as mesylate, tosylate, or triflate).

One can produced [¹⁸F] fluoride by irradiation of water (containing H₂¹⁸O) with protons resulting in the reaction ¹⁸O(p,n)¹⁸F. For production efficiency and radiochemical purity, it is desirable to use water that is as highly enriched as possible. The [¹⁸F] isotope is then separated from water and processed for production of a radiopharmaceutical agent. Typically, fluoride recovery is based on ion exchange resins. The recovery is carried out in two steps (extraction and elution): first the anions (not only fluoride) are separated from the enriched [¹⁸O] water and trapped on a resin and then, said anions, including [¹⁸F] fluoride, are eluted into a mixture containing water, organic solvents, a base, also called activating agent or phase transfer agent or phase transfer catalyst, such as the complex potassium carbonate-Kryptofix 222™ (K₂CO₃—K₂₂₂) or a tetrabutylammonium salt. Kryptofix 222™ is a cyclic crown ether, which binds the potassium ion, preventing the formation of ¹⁸F-KF. Thus, potassium acts as the counter ion of ¹⁸F⁻ to enhance its reactivity but does not interfere with the synthesis. Typical labeling methods use low water content solutions. An evaporation step may follow the recovery of the [¹⁸F]fluoride, e.g., azeotropic evaporation of acetonitrile or other low boiling temperature organic solvent.

Alternatively, the extraction process is performed by passing the [¹⁸F] aqueous solution on a solid support as reported in U.S. Pat. No. 8,641,903. This solid support is typically loaded with a trapping agent, e.g., compound comprising a quaternary amine that is adsorbed on the solid support and allows the [¹⁸F] activity to be trapped because of its positive charge. The solid support is then flushed with a gas or a neutral solvent to remove or push out most of the residual water. The [¹⁸F] is at last eluted in an organic solvent or in a mixture of organic solvents and is usable for the labelling of precursor compounds.

### Florescent and Other Labeling Applications

In certain embodiments, this disclosure relates to a compound having the following formula:

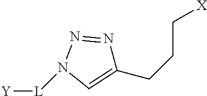

or salts thereof wherein, X is I or Br; and L is a linking group or L is absent and Y is a label, polymer, peptide, glycoprotein, ligand, receptor, antibody, antibody antigen/epitope, steroid, nucleic acid, particle, micelle, vesicle, cell, or solid substrate.

In certain embodiments, this disclosure relates to a compound having the following formula:

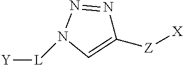

or salts thereof wherein, X is I or Br; Y is a polysaccharide or polysaccharide derivative; and L is a linking group or L is absent and Y forms a direct bond from a sugar unit in the polysaccharide to the nitrogen, and Z is a linking group, —CH₂—, —CH₂CH₂—, —CH₂CH₂CH₂—, —CH₂CH₂CH₂CH₂—, or —CH₂(CH₂)ₙCH₂—, wherein n is 2-20.

Although the examples are provided herein emphasize the stability of the 3-iodopropyl-1,2,3-triazol-1-yl or 3-bromopropyl-1,2,3-triazol-1-yl for radionuclide labeling saccharides, these linking groups can also be used for other labeling strategies and other polymers, particles, biomolecules such as for generating fluorescently labeled saccharides, polysaccharides, peptides, or nucleic acids. Generally, a label can be functionalized with a nucleophilic group such as a primary or secondary amine, i.e., amino group, or a thiol group. The group can act as a nucleophile and react with the 3-iodopropyl-1,2,3-triazol-1-yl or 3-bromopropyl-1,2,3-triazol-1-yl linking groups. The 1,2,3-triazol-1-yl can be formed by the reactions of an azide with triple bonded alkyls, i.e., alkynyls. Proteins can be incorporated with lysine residues that can react with the 3-iodopropyl-1,2,3-triazol-1-yl or 3-bromopropyl-1,2,3-triazol-1-yl linking groups produce labeled proteins. In certain embodiments, the disclosure relates to recombinant polypeptides wherein the amino terminal end of the amino acid sequence is labeled using compounds and procedures reported herein.

A “label” refers to a detectable compound or composition that is conjugated directly or indirectly to another molecule, such as an antibody or a protein, to facilitate detection of that molecule. Specific, non-limiting examples of labels include fluorescent tags, enzymatic linkages, and radioactive isotopes.

A label includes the incorporation of biotinyl moieties to a polypeptide that can be detected by marked avidin (for example, streptavidin containing a fluorescent marker or enzymatic activity that can be detected by optical or colorimetric methods). Various methods of labeling polypeptides and glycoproteins are known in the art and may be used. Examples of labels for polypeptides include, but are not limited to, the following: fluorescent labels (such as fluorescein isothiocyanate (FITC), rhodamine, lanthanide phosphors), enzymatic labels (such as horseradish peroxidase, beta-galactosidase, luciferase, alkaline phosphatase), chemiluminescent markers, biotinyl groups, predetermined polypeptide epitopes recognized by a secondary reporter (such as a leucine zipper pair sequences, binding sites for secondary antibodies, metal binding domains, epitope tags), or magnetic agents, such as gadolinium chelates. In some embodiments, labels are attached by spacer arms of various lengths to reduce potential steric hindrance.

As used herein, the term “ligand” refers to any organic molecule, i.e., substantially comprised of carbon, hydrogen, and oxygen, that specifically binds to a “receptor.” Receptors are organic molecules typically found on the surface of a cell. Through binding a ligand to a receptor, the cell has a signal of the extra cellular environment which may cause changes inside the cell. As a convention, a ligand is usually used to refer to the smaller of the binding partners from a size standpoint, and a receptor is usually used to refer to a molecule that spatially surrounds the ligand or portion thereof. However as used herein, the terms can be used interchangeably as they generally refer to molecules that are specific binding partners. For example, a glycan may be expressed on a cell surface glycoprotein and a lectin protein may bind the glycan. As the glycan is typically smaller and surrounded by the lectin protein during binding, it may be considered a ligand even though it is a receptor of the lectin binding signal on the cell surface. An antibody may be a receptor, and the epitope may be considered the ligand. In certain embodiments, a ligand is contemplated to be a compound that has a molecular weight of less than 500 or 1,000. In certain embodiments, a receptor is contemplated to be a protein-based compound that has a molecular weight of greater than 1,000, 2,000 or 5,000.

### EXAMPLES

### 4-bromophenylsulfonyloxymaltohexaose F-18 Labeling

The production of maltohexaose F-18 derivatives (MHF) using a 4-bromophenylsulfonyloxymaltohexaose F-18 labeling precursor by a remote semi-automated method was reported in Ning et al., Angewandte Chemie Int., 2014, 53:14096-14101. The method was appropriate for preclinical evaluation; however, it was not suitable for the repeated aseptic production of F-18 MHF for human use employing a production, cGMP, device such as the GE TracerLab™ FX N platform. Shortcomings of the 4-bromophenylsulfonyloxy-maltohexaose F-18 labeling precursor made it inappropriate for the cGMP production because of low reaction yields, short shelf life, and instability of the solution at elevated temperatures required for F-18 labeling. These shortcomings lead to a high number of failures, inconsistent, and low radiochemical yields using the remote semi-automated methods.

### 4-Methylphenylsulfonyloxy maltohexaose F-18 Labeling

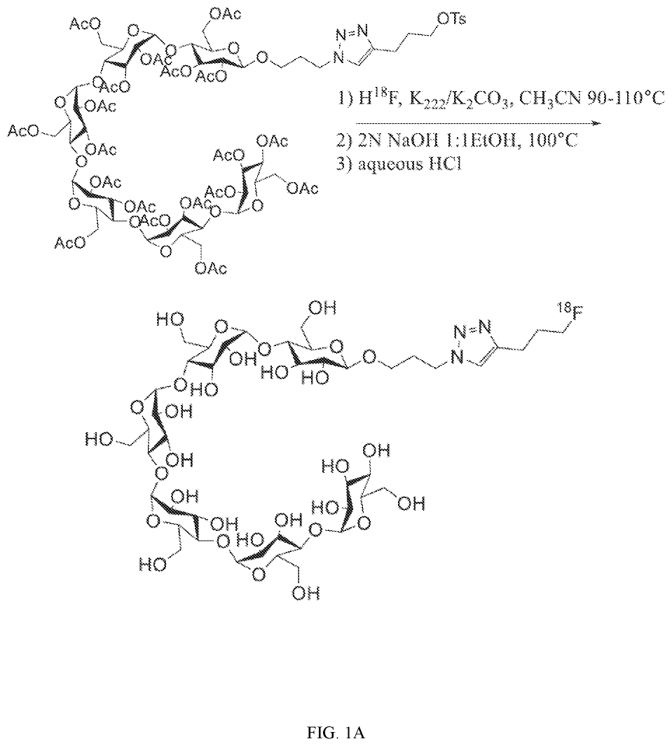

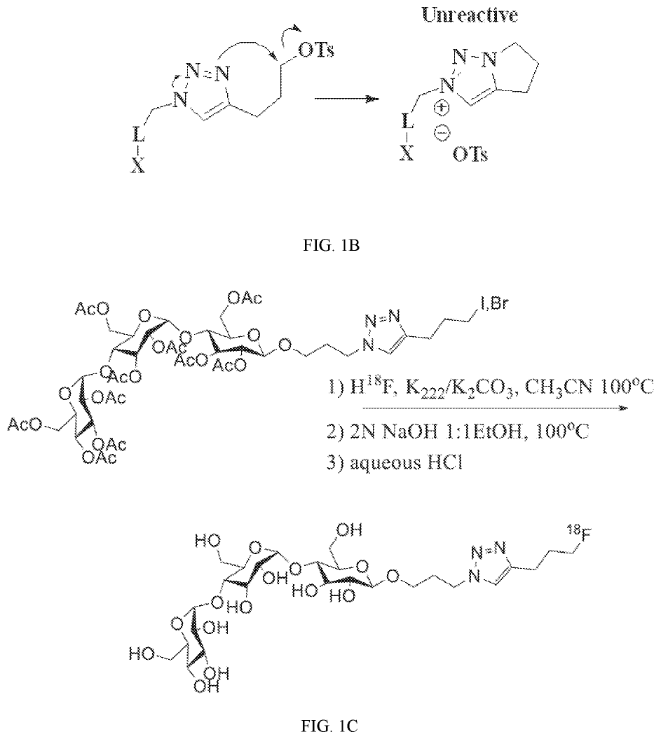

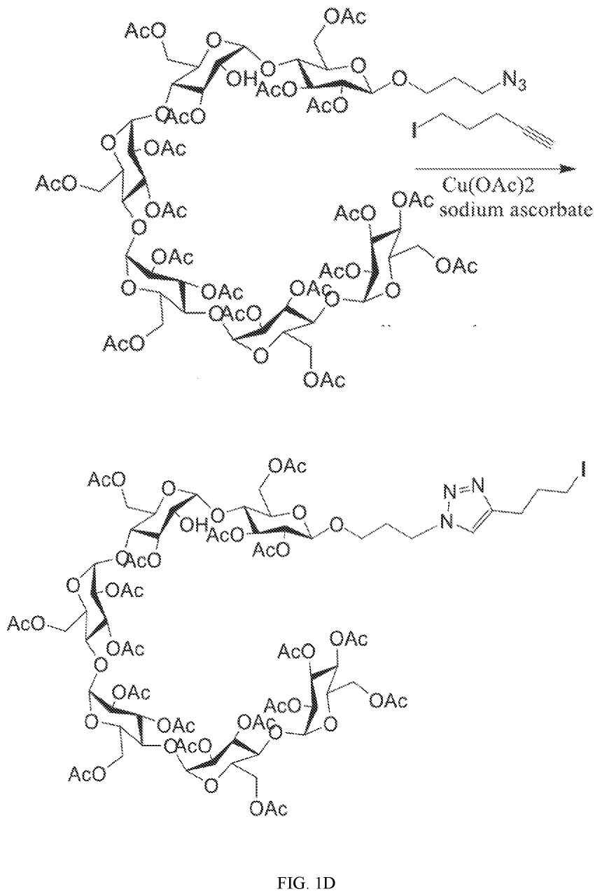

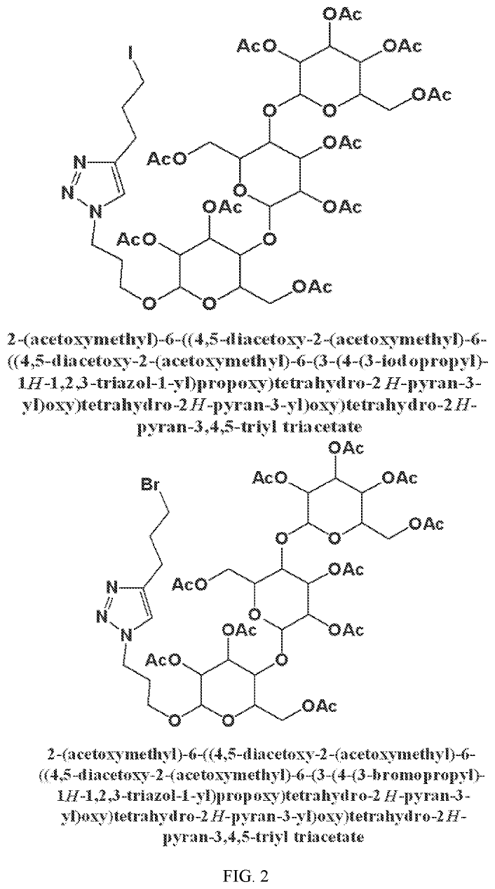

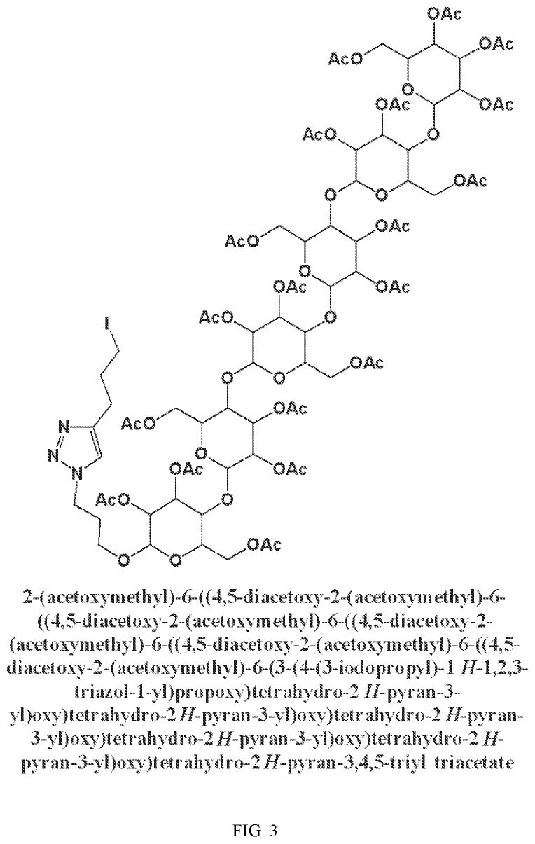

Experiments were performed to determine whether the 4-methylphenylsulfonyloxy (tosylate) precursor could be substituted for the brosylate precursor (p-bromophenylsulfonic acid ester precursor). The tosylate precursor was prepared. See FIG. 1A. The 4-methylphenylsulfonyloxy precursor also afforded inconsistent radiochemical yields of the F-18 MHF product (See table 1). Similar to the brosylate precursor, the tosylate precursor had inconsistent yields and a short shelf life.

Table of F-18 MHF by MH-Tosylate using GE TracerLab FXN2 Pro

| Date | Oct. 15, 2019 | Oct. 18, 2019 | Nov. 5, 2019 | Nov. 7, 2019 | Nov. 15, 2019 | Nov. 19, 2019 | Nov. 22, 2019 |
|---|---|---|---|---|---|---|---|
| NOTEBOOK | WS-8-278 | WS-8-280 | WS-8-291 | WS-8-293 | WS-8-296 | WS-8-297 | WS-8-299 |
| Precursor | 6.1 | 6.0 | 6.1 | 6.1 | 12.2 | 6.2 | 12.1 |
| mg | | | | | | | |
| F-18 Rxn ° C. | 110 | 110 | 90 | 100 | 100 | 100 | 100 |
| mL | 1N | 1N | 1N | 2N | 2N | 2N | 2N |
| 2N NaOH- | 1 + 1 | 1 + 1 | 1 + 1 | 1 + 1 | 1 + 1 | 1 + 1 | 1 + 1 |
| EtOH | | | | | | | |
| Hydrolysis | 105/80 | 105/80 | 90/80 | 100/80 | 100/80 | 100/80 | 100/80 |
| Rxn ° C. | | | | | | | |
| Hydrolysis | 10/5 | 10/5 | 10/5 | 15/5 | 15/5 | 10/5 | 10/5 |
| Rxn min | | | | | | | |
| Prep HPLC | 6% | 6% | 6% | 6% | 6% | 6% | 6% |
| Eluent | EtOH | EtOH | EtOH | EtOH | EtOH | EtOH | EtOH |
| F-18 MHF | n/a | n/a | 20.40 | 19.00 | 19.04 | 21.35 | 16.39 |
| Rt (min · sec) | | | | | | | |
| Prep HPLC | n/a | n/a | Novapak, | Novapak, | Novapak, | Atlantis | Atlantis |
| Eluent | | | MeOH | MeOH | MeOH | T3 EtOH | T3 EtOH |
| (%, v/v) | | | (7.5) | (7.5) | (7.5) | (6.0) | (6.0) |
| F-18 MHF | n/a | n/a | 6.9 | 8.1 | 8.9 | 10.5 | 11.5 |
| Rt(min) | | | | | | | |
| Starting | 1388 | 2058 | 1249(763.2) | 1226(698.9) | 1778(1033) | 1265(754) | 744(457.5) |
| F-18 | | | | | | | |
| Production | n/a | n/a | 2.3 | 3.5 | 43 | 16.7 | 8 |
| Vial | | | | | | | |
| F-18 MHF | n/a | n/a | 0.3 | 0.5 | 4.2 | 2.2 | 1.7 |
| RCY % EOB | | | | | | | |

Although it is not intended that embodiments of this disclosure be limited by any particular mechanism, it is contemplated that the carbon bearing the 4-bromophenylsulfonyloxy and 4-methyphenylsulfonyloxy groups may undergo an intramolecular reaction with the 1,2,3-triazolo group, giving a non-reactive polar products (see FIG. 1B). Side products formed even if the precursor was cooled in a freezer and during the F-18 fluorination reaction at 100 C.

### 3-Iodopropyl and 3-Bromopropyl Triazine Derivatives F-18 Labeling

Experiments were performed to determine whether the use of 3-bromopropyl- and 3-iodopropyl-precursors could be used as a reliable substitute to prepare F-18 labeled maltotriose, MTF (FIG. 1C). The results shown in Table 2 demonstrated that the 3-bromopropyl- and 3-iodopropyl-precursors afforded F-18 MTF in non-optimized reasonable and reproducible radiochemical yields. Both the 3-bromopropyl- and 3-iodopropyl-precursors were stable when stored below 0 C.

TABLE 2
F-18 MTF by MT-Bromo and Iodo precursors using GE TracerLabFXN2 Pro

| Precursor mg | 6.9-MT-I | 7.2-MT-I | 7.5-MT-I | 7.3-MT-Br | 7.2-MT-Br | 7.2-MT-Br |
|---|---|---|---|---|---|---|
| Precursor | 1.0 | 1.0 | 1.0 | 1.0 | 1.0 | 1.0 |
| CH₃CN mL | | | | | | |
| F-18 Rxn ° C. | 100 | 100 | 100 | 100 | 100 | 100 |
| mL | 2N | 2N | 2N | 2N | 2N | 2N |
| 2N | 1 + 1 | 1 + 1 | 1 + 1 | 1 + 1 | 1 + 1 | 1 + 1 |
| NaOH/EtOH | | | | | | |
| Hydrolysis | 100/80 | 100/80 | 100/80 | 100/80 | 100/80 | 100/80 |
| Rxn ° C. | | | | | | |
| Hydrolysis | 10/5 | 10/5 | 10/5 | 10/5 | 10/5 | 10/5 |
| Rxn min | | | | | | |
| Prep HPLC | 9% EtOH | 8% EtOH | 8% EtOH | 8% EtOH | 8% EtOH | 8% EtOH |
| Eluent | | | | | | |
| F-18 MTF Rt | 5.32 | 14.19 | 19.08 | 17.21 | 17.25 | 17.04 |
| (min · sec) | | | | | | |
| Prep HPLC | Atlantis T3 | Atlantis T3 | Atlantis T3 | Atlantis T3 | Atlantis T3 | Atlantis T3 |
| Eluent | EtOH (9.0) | EtOH (8.0) | EtOH (8.0) | EtOH (8.0) | EtOH (8.0) | EtOH (8.0) |
| (%, v/v) | | | | | | |
| Starting | 1242(869.4) | 1294(905.8) | 1349(944.3) | 1263(884.1) | 1332(932.4) | 1315(920.5) |
| F-18 | @10:02 AM | @10:02 AM | @8:58 AM | @8:53 AM | @8:56 AM | @08:54 AM |
| Production | 66.5@11:10 | 55.5@11:19 | 76.6@10:18 | 19.37@10:13 | 32.2@10:17 | 27.5@01:13 |
| Vial | AM | AM | AM | AM | AM | AM |
| F-18 MTF | 11.7 | 9.9 | 13.5 | 3.6 | 6.2 | 4.9 |
| RCY % EOB | | | | | | |

### Synthesis of the iodopropyl precursor MH-OAc-I

The iodopropyl precursor, MH-OAc-I was prepared (FIG. 1D). Beta-MH-OAc-N₃ (1, 0.419 g, 0.2237 mmol, 1 equiv) was dissolved in t-BuOH/CH3CN (12 mL/0.5 mL) and 5-iodo-1-pentyne (0.052 g, 0.2684 mmol, 1.2 equiv) was added. Cu(OAc)2 (0.04 g, 112 μL of 0.2 M aqueous solution, 0.0223 mmol, 0.1 equiv) was added followed by sodium ascorbate (0.088 g, 112 μL of 0.4 M aqueous solution, 0.0447 mmol, 0.2 equiv). After the reaction mixture was stirred at room temperature under a nitrogen atmosphere overnight, it was diluted with methylene chloride (20 mL), filtered and concentrated to remove t-BuOH. The clear yellow oily residue was dissolved in methylene chloride (80 mL), and the organic solution was washed with water (2×30 mL) and brine (1×30 mL), dried over anhydrous Na₂SO₄, filtered and concentrated. The crude product was purified by silica gel column chromatography (ethyl acetate/hexane/methanol from 5:1:0 to 5:1:0.1) to give 2 as a clear, pale yellow oil (0.39 g, 85%).

### Sterile, Pyrogen-Free Solution of No-Carrier-Added [F-18]MHF

The preparation of [F-18]MHF (FIG. 1D)) was carried out in a GE TRACERlab™ FX-N Pro. The radiosynthesis of [F-18]MHF was accomplished via the following procedure: 1) [¹⁸F]fluorination of precursor 2, 2) Basic hydrolysis of [¹⁸F]3 3) HPLC purification of [F-18]MHF, and 4) formulation. Prior to the start of synthesis of [F-18]MHF, appropriate reagent solutions were loaded into five (5) reagent vials on the FX-N Pro module. Vial 1 was used for the elution of F-18 fluoride from the QMA cartridge. Vial 2 was used for the addition of acetonitrile for azeotrpic drying. Vial 3 was used for the addition of the precursor; vials 4 and 5 were used for basic hydrolysis and neutralization, respectively prior to HPLC purification. Vial 6 is empty. Vial 1 was added with fresh 22 mg K₂₂₂ in 0.3 mL acetonitrile, 7 mg K₂CO₃ in 0.3 mL water, Vial 3 was added with the iodide precursor 2 (24 mg) in dry acetonitrile (1 mL); Vial 4 was added with 2N NaOH (1 mL) and USP ethanol (1 mL) and Vial 5 was added with 0.67N HCl (3 mL). [¹⁸F]Fluoride, 2.0 Ci of no-carrier-added [¹⁸F]HF (60 μA, 45 minute bombardment, theoretical specific activity of 1.7 Ci/nmole), produced on a Siemens RDS 111 cyclotron was adsorbed on QMA cartridge. The [¹⁸F]fluoride was eluted from the QMA cartridge with 22 mg K₂₂₂ and 7 mg K₂CO₃ in 0.6 mL 1:1 acetonitrile:water into Reactor 1. 1.0 mL of acetonitrile was added to Reactor 1. The Reactor 1 temperature was raised up to 90° C. for 9 minutes under vacuum with helium flow for solvent evaporation to dryness. The temperature is then raised to 115° C. with vacuum and Helium flow for 4 minutes. MH-OAc-I precursor 2 (24 mg, 0.012 mmol) in 1.0 mL of dry acetonitrile was added from Vial 3. The radiofluorination process was performed at 100° C. for 20 min. Following radiofluorination the Reactor 1 temperature was lowered to 50° C. and solvent evaporation was performed under vacuum for 6 min to dryness. The Reactor 1 temperature was lowered to 45° C. and the basic hydrolysis process was performed by adding 2N NaOH (1 mL) and USP ethanol (1 mL) from Vial 4 followed by heating at 100° C. for 10 minutes and 80° C. for 5 minutes. The hydrolysis at 80° C. was performed under helium gas flow and under vacuum. After basic hydrolysis the Reactor 1 temperature was lowered to 45° C. and the reaction mixture was neutralized by adding 0.67 N HCl (3 mL) from Vial 5. The neutralization solution was injected onto a Waters Atlantis™ T3, 5 μm, 19 mm×100 mm HPLC column. The HPLC purification was done at 6 mL/min using 600 EtOH-water as eluent. The radioactive peak of [F-18]MHF was collected at 16.5-18 min into a flask. Finally, the product in 6-10 mL 6% EtOH-water was sterilized by passing through a Millex™ GV sterile filter into an aseptically prepared dose vial.

TABLE 3
F-18 MHF by MH Iodo precursor using GE TracerLabFXN2

| | | | | |
|---|---|---|---|---|
| K₂₂₂(mg) | 11.0 | 11.0 | 11.0 | 11.0 |
| mL methanol | 1.0 | 1.0 | 1.0 | 1.0 |
| K₂CO₃ mg | 0.8 | 0.8 | 0.8 | 0.8 |
| Water mL | 0.2 | 0.2 | 0.2 | 0.2 |
| F-18 Dry ° C. | 65/95 | 65/95 | 65/95 | 65/95 |
| F-18 Dry min | 3/7 | 3/7 | 3/7 | 3/7 |
| Precursor mg | 13 | 26 | 26 | 24 |
| Precursor | 1.0 | 1.0 | 1.0 | 1.0 |
| CH₃CN mL | | | | |
| F-18 Rxn ° C. | 100 | 100 | 100 | 100 |
| F-18 Rx nmin | 10 | 10 | 20 | 20 |
| mL 2N | 2N1/1 | 2N1 + 1 | 2N1 + 1 | 2N1 + 1 |
| NaOH/EtOH | | | | |
| Hydrolysis | 100/80 | 100/80 | 100/80 | 100/80 |
| Rxn ° C. | | | | |
| Hydrolysis | 10/5 | 10/5 | 10/5 | 10/5 |
| Rxn min | | | | |
| Prep HPLC | 6% EtOH | 6% EtOH | 6% EtOH | 6% EtOH |
| Eluent | | | | |
| F-18 MHFR t | 16.48 | 17.0 | 12.53 | 14.48 |
| (min · sec) | | | | |
| Prep HPLC | AtlantisT3EtOH(6.0) | AtlantisT3EtOH(6.0) | AtlantisT3EtOH(6.0) | AtlantisT3EtOH |
| Eluent | | | | (6.0) |
| (%, v/v) | | | | |
| Starting F-18 | 1470(1029)@10:20AM | 1295(754)@10:06AM | 1116(781.2)@9:59AM | 1230@10:29AM |
| Production | 30.8@11:40AM | 39.9@11:27AM | 47.1@11:25AM | 47.3@11:58AM |
| Vial | | | | |
| F-18 | 5.0 | 7.3 | 10.4 | 8.1% |
| MHFRCY % | | | | |
| EOB | | | | |
| F-18 | 98 | 99 | 99 | 99.5 |
| MHFRCP % | | | | |
| pH | 5.3 | 5.3 | 5.3 | 5.3 |

What is claimed is:

1. A compound having the following formula: 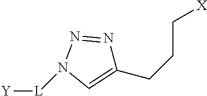
or salts thereof wherein,
X is I or Br;
Y is a polysaccharide or polysaccharide derivative; and
L is a linking group.

2. The compound of claim 1, wherein the polysaccharide derivative is an acetylated polysaccharide of maltotriose, isomaltotriose, maltoheptaose, or maltohexaose.

3. The compound of claim 1, wherein the linking group is an alkyl group or glycol group.

4. The compound of claim 1 which is 2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3-yl)oxy)tetrahydro-2H-pyran-3,4,5-triyl triacetate or salt thereof.

5. The compound of claim 1 which is 2-(acetoxymethyl)-6-((4,5-diacetoxy-2-(acetoxymethyl)-6-((4,5-diacetoxy-2-(acetoxymethyl)-6-(3-(4-(3-iodopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

6. The compound of claim 1 which is 2-(acetoxymethyl)-6-((4,5-diacetoxy-2-(acetoxymethyl)-6-((4,5-diacetoxy-2-(acetoxymethyl)-6-(3-(4-(3-bromopropyl)-1H-1,2,3-triazol-1-yl)propoxy)tetrahydro-2H-pyran-3-yl)oxy)tetrahydro-2H-pyran-3-yl)oxy)tetrahydro-2H-pyran-3,4,5-triyl triacetate or salt thereof.

7. A method of generating a compound isotopically enriched with fluorine 18 comprising contacting a compound as in claim 1 with hydrofluoric acid isotopically enriched with fluorine 18 providing a compound isotopically enriched with fluorine 18.

8. The method of claim 7 further comprising contacting the compound isotopically enriched with fluorine 18 with a deacetylating agent such that alkanoyl groups on compound are converted to hydroxy groups providing a polysaccharide isotopically enriched with fluorine 18.

9. The method of claim 8 wherein the deacetylating agent is a metal hydroxide in an aqueous ethanol solution.

10. A method comprising:
a) administering a composition comprising a polysaccharide or polysaccharide derivative isotopically enriched with fluorine 18 made by the process of claim 7 to a subject; and
b) scanning the subject for emissions.

11. The method of claim 10 further comprises the step of detecting the emissions and creating an image indicating or highlighting the location of the compound containing polysaccharide or polysaccharide derivative isotopically enriched with fluorine 18 in the subject.

12. A kit comprising a compound of claim 1 and potassium bound to [2.2.2]-cryptand.
